(19) United States
(12) Patent Application Publication
(10) Pub. No.: US 2010/0152610 A1
(43) Pub. Date: Jun. 17, 2010

(54) Hand Actuated Tetherless Biopsy Device with Pistol Grip

(76) Inventors: Shailendra K. Parihar, Mason, OH (US); Michael J. Andreyko, Cincinnati, OH (US); Kyle P. Moore, Mason, OH (US); Eric B. Smith, Cincinnati, OH (US); James Janszen, Cleves, OH (US)

Correspondence Address: FROST BROWN TODD, LLC, 2200 PNC CENTER, 201 E. FIFTH STREET, CINCINNATI, OH, 45202, US

(21) Appl. No.: 12/335,578

(22) Filed: Dec. 16, 2008

(51) Int. Cl. A61B 10/02

(52) U.S. Cl. 600/566

(57) ABSTRACT

A hand actuated biopsy device comprises a vacuum generation mechanism and a tissue cutting mechanism manually actuated by a trigger of the hand actuated biopsy device. Hand manipulation or actuation of the trigger is used to power a vacuum pump to generate vacuum and to power the tissue cutting mechanism. The hand actuated biopsy device has a tissue piercing needle with a tissue receiving aperture. A tissue cutter translates and rotates within the needle and is powered by actuations of the trigger. When the needle is placed into tissue and the trigger is actuated to power the vacuum pump and the tissue cutter, tissue is drawn into the tissue receiving aperture and severed by the hand powered cutter. A tissue collection chamber can be attached to the hand actuated biopsy device to receive severed tissue samples within.

### BACKGROUND

Biopsy samples have been obtained in a variety of ways in various medical procedures using a variety of devices. Biopsy devices may be used under stereotactic guidance, ultrasound guidance, MRI guidance, or otherwise. Merely exemplary biopsy devices are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pub. No. 2003/0109803, entitled “MRI Compatible Surgical Biopsy Device,” published Jun. 12, 2003; U.S. Pub. No. 2007/0118048, entitled “Remote Thumbwheel for a Surgical Biopsy Device,” published May 24, 2007; U.S. Pub. No. 2008/0214955, entitled “Presentation of Biopsy Sample by Biopsy Device,” filed Nov. 20, 2007; U.S. Provisional Patent Application Ser. No. 60/869,736, entitled “Biopsy System,” filed Dec. 13, 2006; and U.S. Provisional Patent Application Ser. No. 60/874,792, entitled “Biopsy Sample Storage,” filed Dec. 13, 2006. The disclosure of each of the above-cited U.S. patents, U.S. patent application Publications, and U.S. Provisional Patent Applications is incorporated by reference herein. While several systems and methods have been made and used for obtaining a biopsy sample, it is believed that no one prior to the inventors has made or used the invention described in the appended claims.

### BRIEF DESCRIPTION OF THE FIGURES

While the specification concludes with claims which particularly point out and distinctly claim the biopsy device, it is believed the present biopsy device will be better understood from the following description of certain examples taken in conjunction with the accompanying drawings, in which like reference numerals identify the same elements and in which:

FIG. 1 is a left side view of an exemplary biopsy device;

FIG. 2 is a side cross sectional view of a needle portion of the biopsy device of FIG. 1;

FIG. 3 is a perspective view of the distal end of a cutter of the needle portion of FIG. 2;

FIG. 4 is a left side view of the biopsy device of FIG. 1 with a left cover removed to show a tissue cutting mechanism and a vacuum generation mechanism;

FIG. 5 is a left side view of a hand actuated vacuum pump assembly of the biopsy device of FIG. 1 in an un-actuated position;

FIG. 6 is a left side view of the hand actuated vacuum pump assembly of the biopsy device of FIG. 1, in an actuated position;

FIG. 7 is a right side view of the biopsy device of FIG. 1 with a right cover removed and the trigger in an un-actuated position;

FIG. 8 is an exploded view of the drive shift mechanism of the biopsy device of FIG. 1;

FIG. 9 is a right side view of the drive shift mechanism of FIG. 8, with the drive shift mechanism in an initial position configured to advance the cutter distally;

FIG. 10 is a right side view of the drive shift mechanism of FIG. 8, with the drive shift mechanism in position configured to retract the cutter proximally;

FIG. 11 is a side cross-sectional view of a needle portion and an auto pressure regulator of the biopsy device of FIG. 1, with the auto pressure regulator open to admit atmospheric pressure air into the needle portion;

FIG. 12 is a side cross-sectional view of the needle portion and auto pressure regulator of FIG. 11, with the auto pressure regulator moved proximally to prevent atmospheric pressure air from entering into the into the needle portion;

FIG. 13 is a side cross-sectional view of an exemplary alternative needle portion; and

FIG. 14 is a perspective view of the needle portion of FIG. 13.

### DETAILED DESCRIPTION

The following description of certain examples of the biopsy device should not be used to limit the scope of the present biopsy device. Other examples, features, aspects, embodiments, and advantages of the biopsy device will become apparent to those skilled in the art from the following description, which is by way of illustration, one of the best modes contemplated for carrying out the biopsy device. As will be realized, the biopsy device is capable of other different and obvious aspects, all without departing from the spirit of the biopsy device. Accordingly, the drawings and descriptions should be regarded as illustrative in nature and not restrictive.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

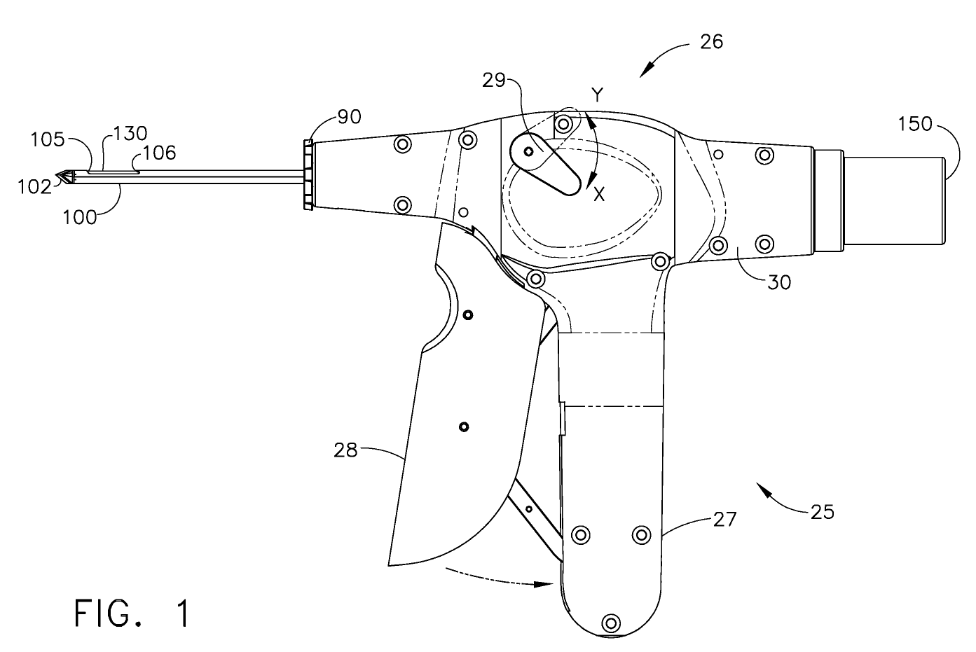

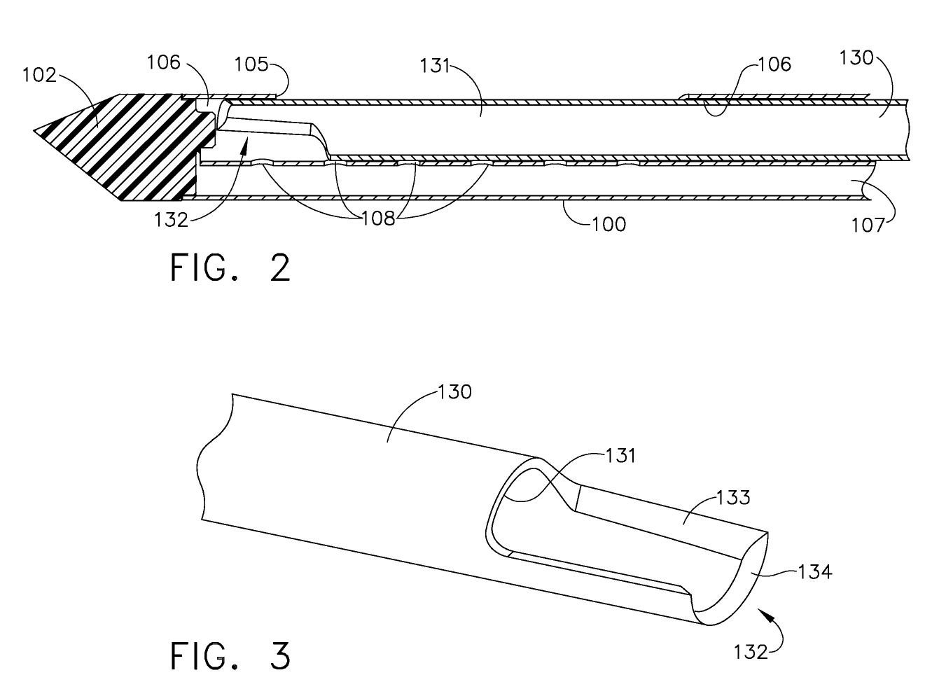

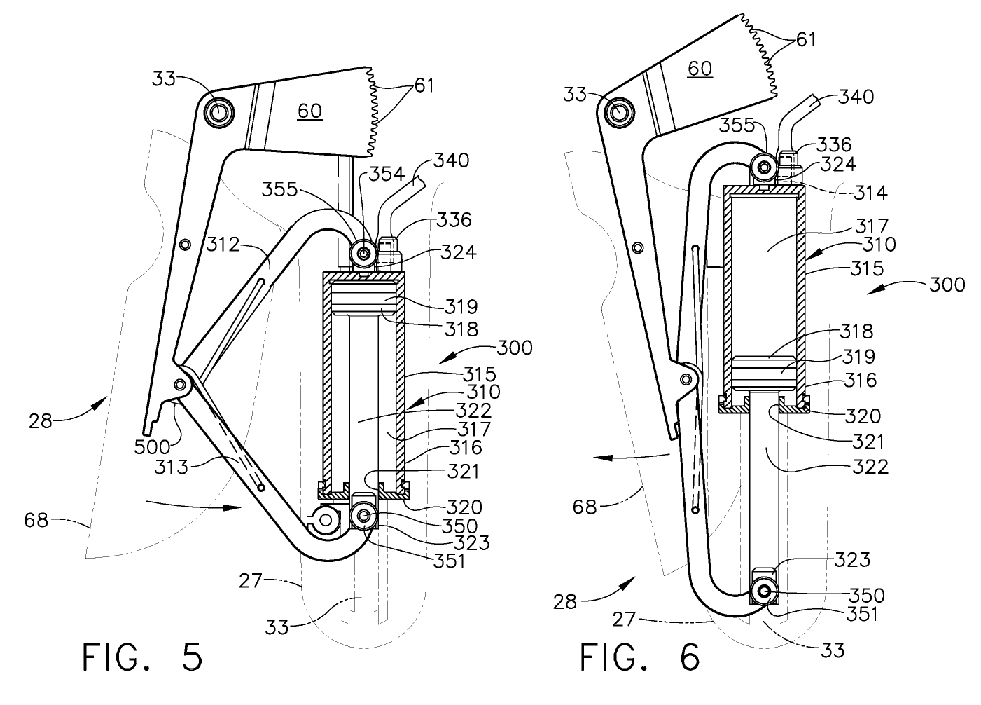

FIG. 1 shows an exemplary biopsy device (25) that is small enough to be hand held, is entirely self contained, and can collect one or more biopsy tissue samples from a patient. Biopsy device (25) comprises an exemplary tissue cutting mechanism (200) (FIGS. 2-4) and an exemplary vacuum generating mechanism (300) (FIGS. 5-6) that are powered by one or more movements of an operator's hand to capture, cut, transport, and collect tissue samples from a patient, such as from a patient's breast.

Hand powered biopsy device (25) of the present example comprises a body (26) with a pistol grip (27), and a manually actuatable trigger (28). A rotatable needle portion (100) defines a longitudinal axis of biopsy device (25) and extends distally from body (26). As shown in FIG. 2, needle portion (100) comprises a distal tissue piercing tip (102) and a hollow cutter passage (106) extending proximally therefrom into body (26). A tissue receiving aperture (105) is located proximal from piercing tip (102) and opens into hollow cutter passage (106) for tissue capture therein. A hollow cutter (130) of the tissue cutting mechanism (200) is slidably and rotatably positioned within cutter passage (106) to cut tissue drawn into aperture (105). Cutter (130) extends through biopsy device (25), from needle portion (100) and through body (26), to operably connect with a tissue collection chamber (150) removably attached to a proximal end of body (26). Tissue samples can be cut with cutter (130) and then drawn through hollow cutter (130) and into tissue collection chamber (150) with vacuum mechanism (300) as will be described in greater detail below. A directional reversal lever (29) is located on each side of biopsy device (25) to reverse directional movement of cutter (130) as tissue samples are acquired. As shown, directional reversal lever (29) has a first downward position X and a second upward position Y.

### I. Exemplary Needle Portion

FIG. 2 shows a cross sectional view of the exemplary rotatable needle portion (100) of biopsy device (25), showing distal tissue piercing tip (102) and hollow cutter passage (106), in which cutter (130) is slidably disposed. The exemplary rotatable needle portion (100) rotatably attaches to body (26) and is rotatable about its longitudinal axis, relative to body (26). Hollow cutter (130) is shown slidably and rotatably mounted in hollow cutter passage (106), with a distal cutting end (132) adjacent to distal tissue piercing tip (102). A lateral vacuum passage (107) extends longitudinally in parallel with hollow cutter passage (106) and operably connects with hollow cutter passage (106) via a plurality of vacuum passages (108) extending therebetween. Vacuum passages (108) are provided to assist in drawing tissue into tissue aperture (105) and into hollow cutter passage (106) when hollow cutter (130) is retracted proximally, and when vacuum is applied to lateral passage (107). It should be understood, however, that these features of needle portion (100) are merely exemplary, and that needle portion (100) may be modified in any suitable fashion.

A perspective view of a scoop shaped distal cutting end (132) of hollow cutter (130) is shown in FIG. 3. Scoop shaped distal cutting end (132) comprises a side cutting edge (133) and a distal cutting edge (134) to cut tissue. The cutting angle for cutting edges (133, 134) may be specified as per material properties and rotation speed of cutter (130), as know through available databases or standards. Alternatively, the cutting angle for cutting edges (133, 134) may be specified based on any other factors or in any other suitable fashion. A lumen (131) extends longitudinally through hollow cutter (130). In operation, hollow cutter (130) rotates and translates to sever a tissue sample with distal cutting edge (134) cutting tissue with translational motion and side cutting edge (133) cutting tissue with rotational motion. The cut or severed tissue sample is captured within lumen (131) adjacent to cutting end (132), and may be communicated proximally through lumen (131) to reach tissue collection chamber (150). Of course, alternative versions of cutter (130) may have a variety of alternative features and configurations, if desired.

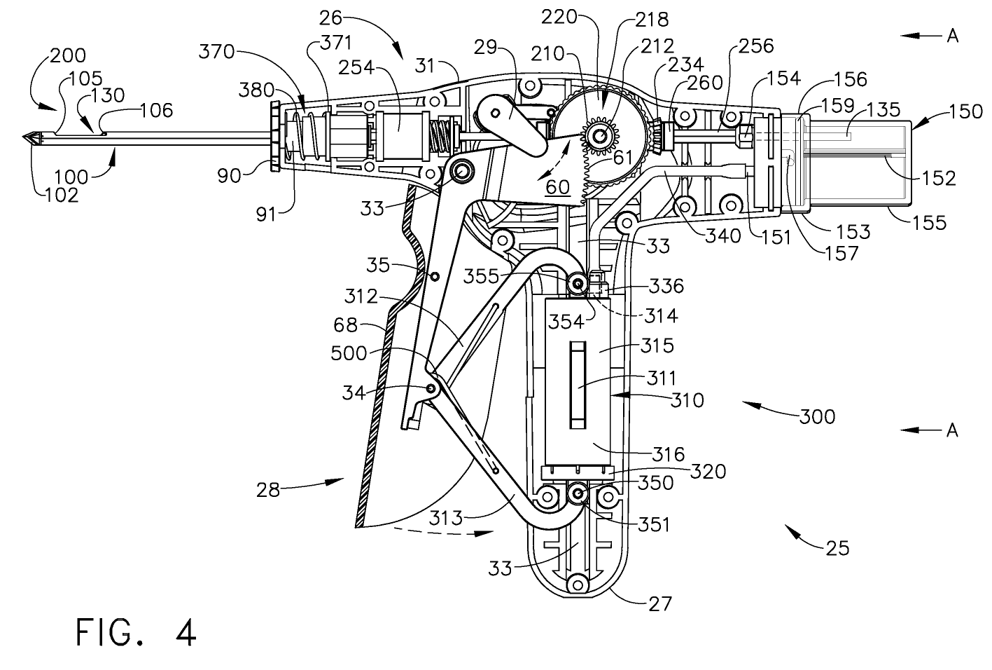

As shown in FIGS. 1 and 4, a knurled knob (90) is fixedly attached to a proximal portion of needle portion (100), and is rotatably attached to body (26) so that rotation of knob (90) rotates needle portion (100) about its longitudinal axis, relative to body (26). Knob (90) has a cylindrical body member (91) extending proximally from knob (90) to a proximal end of needle portion (100). A proximal end of cylindrical body member (91) is open to expose lateral passage (107) and hollow cutter passage (106) and to allow the proximally extending cutter (130) to extend proximally therefrom. Knob (90) and rotatable needle portion (100) are prevented from longitudinal movement by rotational engagement with the covers (30, 31) of body (26). In other versions, however, needle portion (100) may be configured to translate. By way of example only, a spring-loaded, motorized, or other type of firing mechanism may be included to effect translational movement of needle portion (100) relative to body (26). It should also be understood that needle portion (100) need not necessarily be rotatable relative to body (26) in all versions.

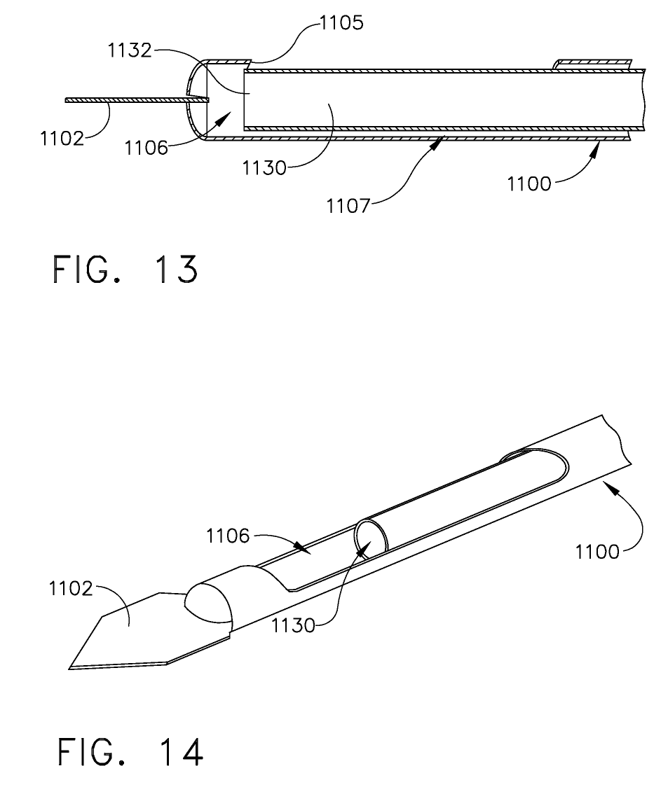

An exemplary alternative needle portion (1100) is shown in FIGS. 13-14. In particular, needle portion (1100) comprises an integrated distal tissue piercing tip (1102) and a hollow cutter passage (1106). A cutter (1130) is slidably disposed in hollow cutter passage (1106). Integrated piercing tip (1102) of the present example is formed by flattening the distal end of needle portion (1100), then grinding away the top layer of the flattened material (leaving only one wall thickness of material), and then grinding the remaining flattened material to form a blade (this blade may take on various shapes). Rotatable needle portion (1100) of this example rotatably attaches to body (26), and is rotatable about its longitudinal axis, relative to body (26). Hollow cutter (1130) is shown slidably and rotatably mounted in hollow cutter passage (1106), with a distal cutting end (1132) adjacent to integrated distal tissue piercing tip (1102). Vacuum is applied through the cutter (1130) in order to prolapse tissue into the receiving aperture (1105). A gap between the outer diameter of cutter (1130) and inner diameter of needle (1100) extends longitudinally in parallel with hollow cutter passage (1106), and communicates venting to cutter (1130) in order to create a pressure differential to transport tissue samples proximally through cutter (1130). It should be understood, however, that these features of needle portion (1100) are merely exemplary, and that needle portion (1100) may be modified in any suitable fashion.

### II. Exemplary Body Portion

FIG. 4 shows the internal elements of the biopsy device (25) of the present example with a left cover (30) removed to expose a right cover (31) of the body (26) positioned relative to other parts. Trigger (28) is pivotally attached to body (26) by a trigger pin (33) that extends from right cover (31). Trigger (28) comprises a rigid inner trigger member (60) fixedly attached to an outer trigger shell (68) by a vacuum pin (34) and a shell pin (35). Trigger shell (68) is ergonomically shaped for the operator to grasp, and is shaped to nest around pistol grip (27) when trigger (28) is moved to a fully actuated position adjacent to pistol grip (27) (FIG. 6). In FIG. 4, trigger shell (68) is sectioned vertically to show the attached inner “L” shaped inner trigger member (60). Trigger member (60) has a downward extending portion shielded by the outer trigger shell (68), and a proximally extending portion with a plurality of gear teeth (61) on a free end thereof. Gear teeth (61) are arrayed in a circular arc around trigger pin (33).

The exemplary tissue cutting mechanism (200) is engaged with trigger (28) via gear teeth (61), and the exemplary vacuum generating mechanism (300) operably engages with trigger (28) via vacuum pin (34). Manual actuation of trigger (28) from an open position (shown in FIGS. 1 and 4) to a closed position (shown in FIG. 6) against pistol grip (27) powers both tissue cutting mechanism (200) and vacuum generation mechanism (300), as will be described in greater detail below.

A. Exemplary Hand Powered Vacuum Generation Mechanism

In the present example as shown in FIGS. 4-7, the exemplary vacuum generation mechanism (300) of hand held biopsy device (25) creates vacuum in response to one or more hand actuations of trigger (28). The vacuum created from movement of the operator's hand is used to draw tissue into tissue aperture (105) of needle portion (100), to hold the drawn tissue within needle portion (100) as a biopsy tissue sample is cut from the held tissue, and to draw the severed biopsy tissue sample through lumen (131) of cutter (130) and into tissue collection chamber (150).

1. Exemplary Vacuum Pump

As shown in FIGS. 4-7, the vacuum generation mechanism (300) comprises a hand actuated vacuum pump (310) that generates vacuum in response to an operator's manual activation of trigger (28). In the present example, vacuum pump (310) can generate 18-20 inches of mercury (inch-Hg) of vacuum (negative pressure) with one or more actuations of trigger (28). Of course, vacuum pump (310) may alternatively generate any other desired amount of pressure at any desired rate. As shown in FIG. 4, vacuum pump (310) is mounted within pistol grip (27) of body (26), and is operably connected to trigger (28) by the vacuum pin (34). Vacuum pump (310) has a pump body (315) with one or more vertical ribs (311) that slidably engage with one or more vertical grooves (33) and/or protrusions within pistol grip (27) to constrain pump (310) in the horizontal direction while allowing movement of pump (310) in the vertical direction. An upper pump arm (312) and a lower pump arm (313) comprise rigid arms that are pivotally attached to a top and a bottom (respectively) of pump body (315), and pump arms (312, 313) are pivotally connected to trigger (28) by vacuum pin (34).

FIGS. 5-6 show cross sectional views of pump (310), in series, as trigger (28) moves from an unactuated position (FIG. 5) to an actuated position (FIG. 6). For clarity, only vacuum pump (310) and the inner trigger member (60) are shown solid, and dashed outlines are provided to show outer trigger shell (68), pistol grip (27), and vertical groove (33) within pistol grip (27). As described previously, vacuum pump (310) is slidingly secured within pistol grip (27) by engagement of vertical ribs (311) within vertical grooves (33) of pistol grip (27), though any other suitable structures or relationships may be employed.

As shown in FIGS. 5-6, pump body (315) further comprises a cylinder portion (316) in vertical orientation, with a hollow bore (317) therein. Cylinder portion (316) has an open end and a closed end. A cylindrical piston (318) is positioned within bore (317) and is movable up and down within. A seal (319) is secured around piston (318) and forms a fixed airtight seal with the piston (318), and a sliding airtight seal with bore (317). A piston rod (322) extends downwardly from a center of piston (318) and passes through an opening (321) within a cover (320) that is fixedly attached to the open end of cylinder (316). A lower connector tang (323) extends downward from a free end of piston rod (322) and has a pin (350) extending therethrough. A proximal end of lower pump arm (313) pivotally attaches to pin (350), and rollers (351) mount on ends of pin (350) outboard of lower connector tang (323) and the proximal end of lower pump arm (313). Rollers (351) are configured to rotate within and to be guided by vertical grooves (33).

An upward connector tang (324) can be located at the top of the pump body (315) to receive pin (354). A proximal end of upper pump arm (312) piviotally attaches to pin (354), and rollers (355) mount on pin (354) outboard of upward connector tang (324) and proximal end of upper pump arm (312). Rollers (355) are configured to rotate within and to be guided by vertical grooves (33).

A spring such as a torsion spring (500) can be placed around vacuum pin (34), with a first spring arm secured to lower pump arm (313) and a second spring arm secured to upper pump arm (312). In the present example, activation of trigger (28) pivots lower pump arm (313) and upper pump arm (312) around pin (34) in a spreading motion, as shown in FIG. 6, and also spreads the first spring arm and second spring arm of torsion spring (500). When trigger (28) is released, the spread torsion spring (500) biases lower pump arm (313), upper pump arm (312), and vacuum pump (310) back to the position shown in FIG. 4.

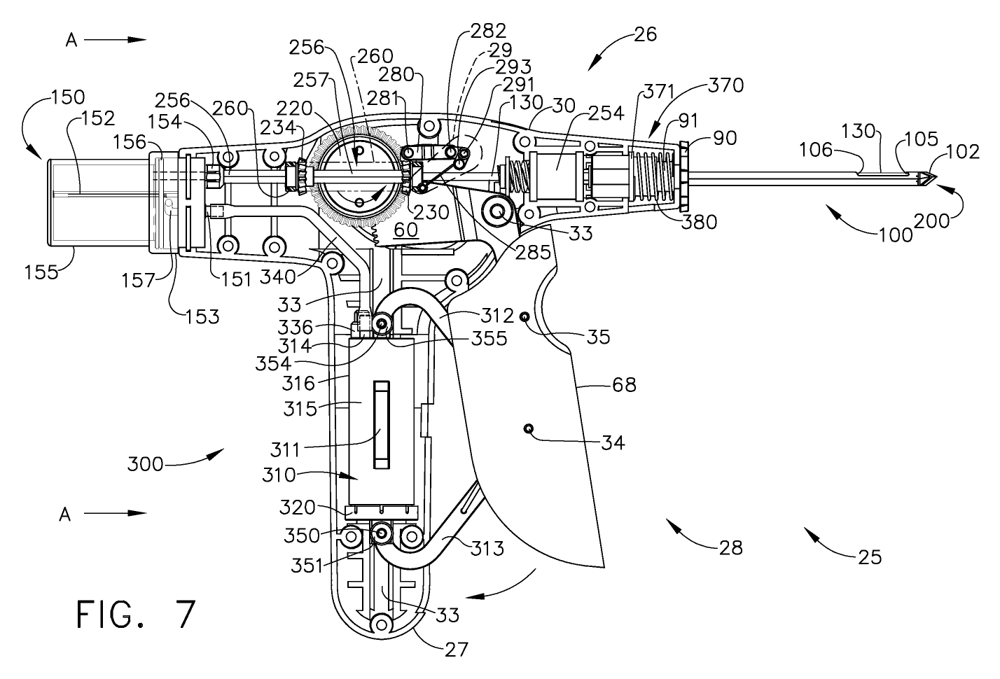

A one way check valve or duck bill valve (336) is attached to the top of pump body (315), and is in fluid communication with bore (317). Duck bill valve (336) opens to atmosphere as piston (318) moves up to purge unwanted air from the bore (317), and closes when piston (318) moves down to draw a vacuum (negative pressure). A flexible hose (340) extends from a top of a nipple (314) and provides fluid communication from bore (317) of pump 310 to a vacuum port (151) of tissue collection chamber (150) (FIGS. 4 and 7) for the induction of a vacuum therein. Flexible hose (340) is configured to bend and move as vacuum pump (310) moves up and down when actuated and deactuated.

In FIG. 6, trigger (28) has been actuated to move cylinder (316) of pump body (315) upwardly and piston (318) and piston rod (322) downwardly to generate a vacuum therebetween. Pump body (315) is guided upwardly by the engagement of vertical ribs (311) in vertical grooves (33), and piston arm (322) is guided downwardly by clevis boss (323) within vertical grooves (33). As shown in FIGS. 5-6, and as noted above, flexible hose (340) bends as pump body (315) moves up and down in response to actuations of trigger (28).

Those of ordinary skill in the art will appreciate that vacuum generation mechanism (300) may be modified, supplemented, or substituted in a variety of ways. By way of example only, while cylinder (316) and piston (318) both move relative to body (26) when vacuum generation mechanism (300) is actuated, other versions may prevent movement of cylinder (316) or piston (318) relative to body (26) when vacuum generation mechanism (300) is actuated. As another merely exemplary alternative, a vacuum generation mechanism (300) may be actuated by something other than a trigger (28). Other suitable components, features, configurations, and methods of operating a vacuum generation mechanism (300) will be apparent to those of ordinary skill in the art in view of the teachings herein.

2. Exemplary Tissue Collection Chamber

Turning back to FIG. 4, and as noted above, tissue collection chamber (150) of the present example is operatively connected to vacuum pump (310) by flexible hose (340). Tissue collection chamber (150) is a hollow assembly that is vacuum tight and comprises a collection base (153) fixedly attached to body (26), and a tissue sample cover (155) that is removably attached to collection base (153), with a vacuum seal (159) therebetween. Collection base (153) and tissue sample cover (155) define at least one hollow volume within that can act as a vacuum accumulator for multiple actuations or pumps of vacuum pump (310). Tissue collection chamber (150) further comprises a tissue collection grid (152) extending within to receive tissue samples within. Tissue collection grid (152) may be configured to permit fluids to pass through grid (152) while preventing tissue samples from passing through grid (152). Tissue collection grid (152) may thus serve as a strainer or filter. Of course, as with other components described herein, tissue collection grid (152) may be modified, substituted, supplemented, or omitted, as desired.

Cutter (130) is operatively engaged with collection base (153) with a seal port (154) that is configured to maintain a vacuum seal with the rotatable and translatable cutter (130), even as cutter (130) rotates and translates relative to seal port (154). Vacuum generated by vacuum pump (310) is delivered to central lumen (131) of cutter (130). In other words, vacuum pump (310) may induce a vacuum within cutter lumen (131) via hose (340) and tissue collection chamber (150). Alternatively, vacuum pump (310) (or any other device) may induce a vacuum within cutter lumen (131) via any other suitable component(s) and/or route(s). In still other versions, a vacuum is simply not induced in cutter lumen (131).

Collection base (153) of the present example further comprises a proximal sample base (156) that releasably holds tissue sample cover (155) onto collection base (153). In particular, collection base (153) presents one or more outwardly extending bayonet pins that are configured to engage with one or more bayonet receivers (157) of tissue sample cover (155). Tissue sample cover (155) is released from collection base (153) by rotation of tissue sample cover (155) relative to collection base (153). Of course, tissue sample cover (155) may be selectively secured relative to body (26) using any other suitable structures, features, or techniques.

In operation, tissue cutter (130) both rotates (around the longitudinal axis) and translates (along the longitudinal axis) during the cutting and acquisition of biopsy tissue samples, and vacuum is used to draw the severed tissue from the vicinity of tissue aperture (105), through lumen (131), and into the tissue collection chamber (150). Cutter (130) has a movable proximal end (135) that is located near the top of tissue sample cover (155) to deliver tissue samples (drawn by vacuum) from proximal end (135) and onto tissue collection grid (152). Tissue collection chamber (150) is thus configured to receive and store the tissue samples on the tissue collection grid (152) as they are transferred (drawn by vacuum) from the proximal end (135) of the tissue cutter (130) and into the tissue collection chamber (150).

Those of ordinary skill in the art will appreciate that tissue collection chamber (150) may be modified, supplemented, or substituted in a variety of ways. Other suitable components, features, configurations, and methods of operating tissue collection chamber (150) will be apparent to those of ordinary skill in the art in view of the teachings herein.

3. Exemplary Auto Pressure Differentiator

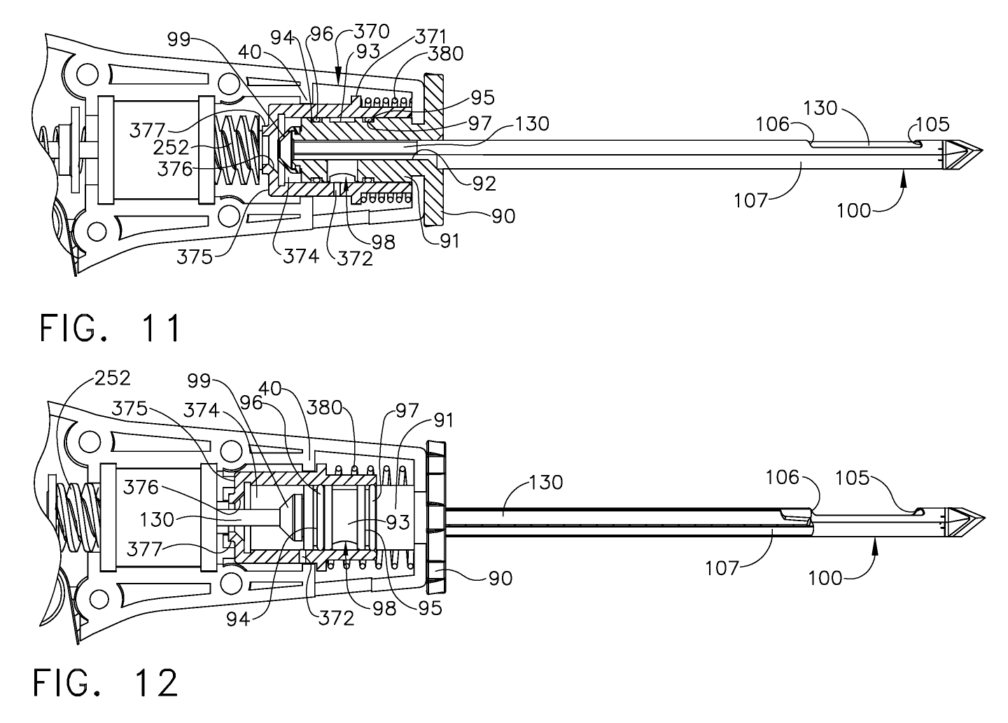

As shown in FIGS. 11-12, an exemplary auto pressure regulator (370) is located at a distal end of body (26) and is configured to longitudinally slide on cylindrical body member (91) of knob (90) and to interact therewith. During manual actuation of biopsy device (25), auto pressure regulator (370) operably couples lateral passage (107) of needle portion (100) to atmospheric pressure during some portions of the tissue acquisition process, and operably decouples the lateral passageway (107) from atmospheric pressure during other portions of the tissue acquisition process. In other words, auto pressure regulator (370) of the present example is operable to selectively provide venting of lateral passage (107).

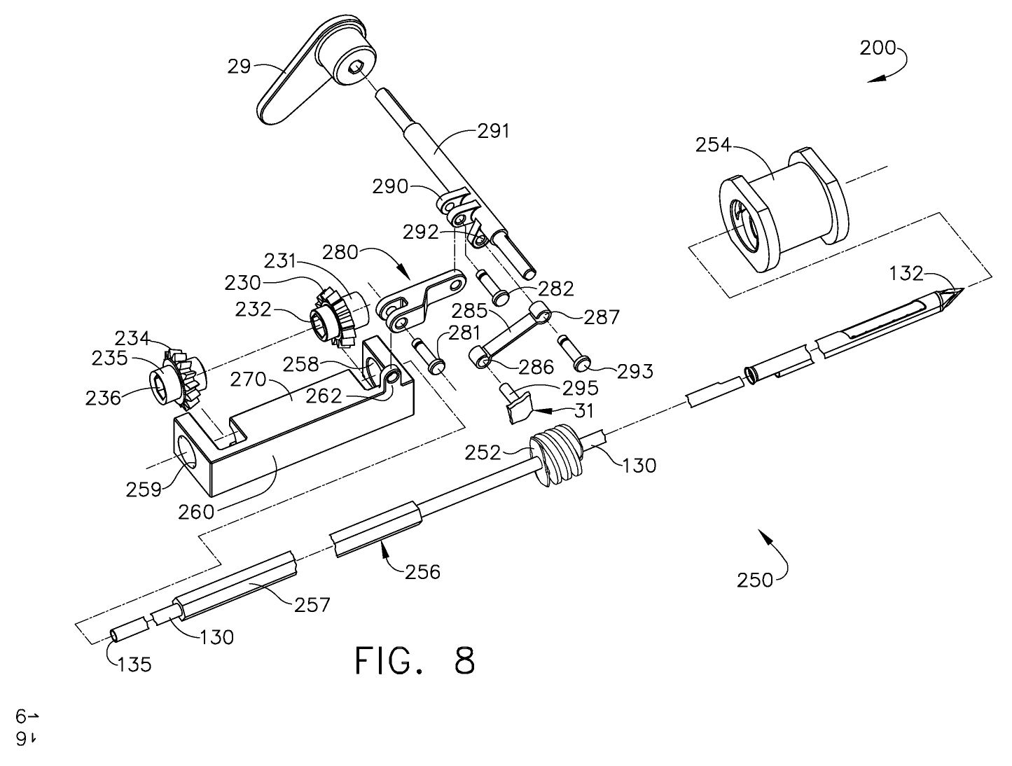

As best shown in FIGS. 11-12, cylindrical body member (91) has an inner bore (92) (with a “FIG. 8” type of cross section) extending longitudinally therethrough, with a proximal end of inner bore (92) forming an airtight seal with needle portion (100). Inner bore (92) is open to both lateral vacuum passage (107) and the hollow cutter passage (106) extending along needle portion (100). A frusto-conical seal (99) is located at a proximal end of open inner bore (92) and is configured to form a rotating and sliding air tight seal with cutter (130). As shown, cutter (130) extends through frusto-conical seal (99), into inner bore (92) and into hollow cutter passage (106) of needle portion (100). An exterior of cylindrical body member (91) has a proximal seal groove (94) and a distal seal groove (95) with a “U” shaped central groove (93) therebetween. Grooves (93, 94, 95) extend around cylindrical body member (91), and an open passageway (98) extends inwardly from central groove (93) to connect with open inner bore (92). An open connection exists between central groove (93), open passageway (98), inner bore (92), lateral vacuum passage (107), and hollow cutter passage (106). An o-ring or proximal elastomeric seal (96) is secured in proximal seal groove (94); and a distal elastomeric seal (97) is secured in distal seal groove (95).

Auto pressure regulator (370) further comprises a cylinder that has an inner bore (374) configured to slidingly receive cylindrical body member (91) within. Inner bore (374) is open at a distal end and has a wall (375) at a proximal end, with a tapered bore (376) extending through wall (375), and a boss (377) for passage of tissue cutter (130) therethrough. Elastomeric seals (96, 97) of cylindrical body member (91) slidingly engage with inner bore (374) to form substantially airtight seals therewith, and to seal or isolate portions of cylindrical body member (91) and central groove (93) therebetween. A centrally located air passage (372) extends through auto pressure regulator (370) and connects with inner bore (374). Auto pressure regulator (370) also has a central flange (371) that engages with a compressible spring (380) to normally bias flange (371) proximally against a rib (40) of body (26) (FIG. 12).

As shown in FIG. 12, when auto pressure regulator (370) is biased proximally against rib (40), cylindrical body member (91) is located in a distal portion of inner bore (374), with distal elastomeric seal (97) just inside the open distal end of inner bore (374) and with proximal elastomeric seal (96) distal to the centrally located air passage (372). In this biased position, open passageway (98) of the cylindrical body member (91) is sealed between seals (96, 97) and inner bore (374) (proximal to cylindrical body member (91)) is communicating with atmospheric air (e.g., vented) through air passage (372) and tapered bore (376).

In FIG. 11, tissue cutting mechanism (200) has been actuated to advance cutter (130) distally. An externally threaded screw (252) is attached around cutter (130) and has advanced distally to contact boss (377) and to push auto pressure regulator (370) distally to the position of FIG. 11. In this position, air passage (372) communicating with inner bore (374) has moved distally past proximal elastomeric seal (96) of cylindrical body member (91) and now communicates air at atmospheric pressure to central groove (93), open passageway (98), inner bore (92), and lateral vacuum passage (107). Lateral vacuum passage (107) is thus vented in this configuration and creates a pressure differential across severed tissue with vacuum induced in lumen (131) of cutter (130). This connection of air at atmospheric pressure to lateral vacuum passage (107) will be discussed further below.

Of course, auto pressure regulator (370) described herein is but one example of many possible structures or features of biopsy device (25). It will be appreciated by those of ordinary skill in the art in view of the teachings herein that the components, features, configurations, and methods of operation of auto pressure regulator (370) may be varied in numerous ways. Furthermore, auto pressure regulator (370) may be omitted altogether in some versions of biopsy device (25).

B. Exemplary Hand Powered Tissue Cutting Mechanism

As previously described, tissue cutting mechanism (200) comprises a hollow cutter (130) that is slidably and rotatably powered by one or more movements of trigger (28) by an operator's hand. Hollow cutter (130) extends longitudinally throughout biopsy device (25), from piercing tip (102) (FIG. 2) to tissue sample container (155), and rotates and translates in response to manual actuations of the trigger (28). The rotational and translational movement of hollow cutter (130) is used to sever tissue samples. The direction of rotation and the direction of translation of the cutter (130) are operator selectable with the previously mentioned directional reversal lever (29).

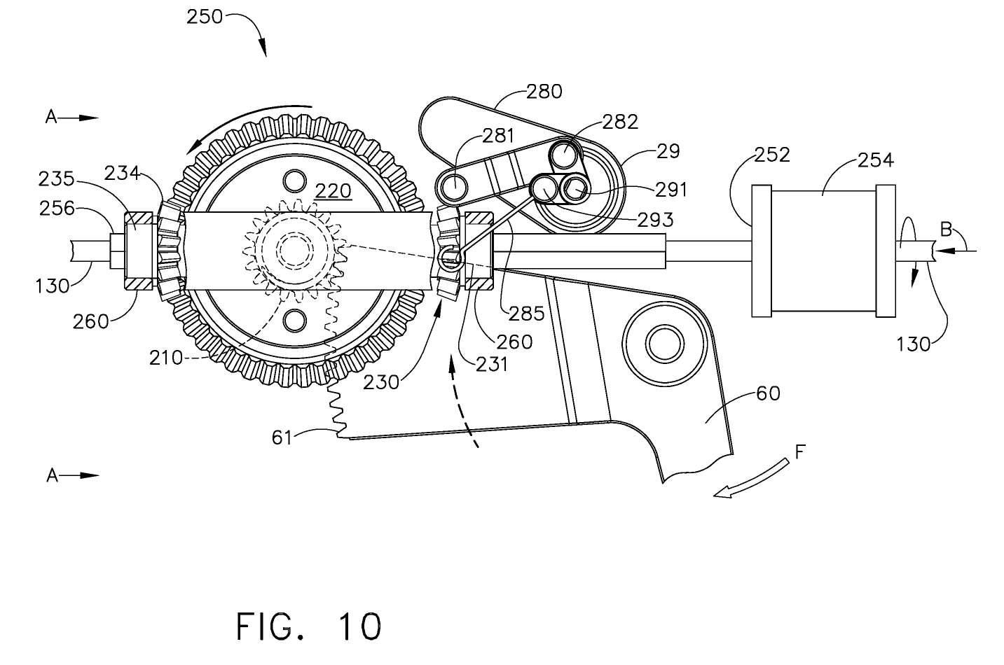

Tissue cutting mechanism (200) of the present example is shown in FIGS. 4 and 7-10. Directional reversal lever (29) is in the first position “X” (or downward position) as shown in FIGS. 1, 4, 7, and 9. When directional reversal lever (29) is in the first position X and trigger (28) is actuated, tissue cutting mechanism (200) translates the cutter (130) proximally along the longitudinal axis to open tissue aperture (105) and rotates the cutter (130) in a non-cutting counter clockwise direction around the longitudinal axis. A second position “Y” (or upwards position) of directional reversal lever (29) is shown as dashed lines in FIG. 1 and as solid lines in FIG. 10. When trigger (28) is actuated with directional reversal lever (29) in the second position, the directions of cutter (130) translation and rotation are reversed so that cutter (130) translates distally along the longitudinal axis to close tissue aperture (105) and to sever tissue; and rotates in a tissue cutting clockwise direction. All indicated clockwise and counterclockwise rotations of cutter (130) are described as if the observer is viewing biopsy device (25) in the direction as indicated by arrows A-A. Of course, the rotational directions described herein could be reversed if desired. Furthermore, in some versions, cutter (130) may simply not rotate at all. For instance, a non-rotating cutter (130) may lack the scoop-like distal end configuration that is shown in the present example.

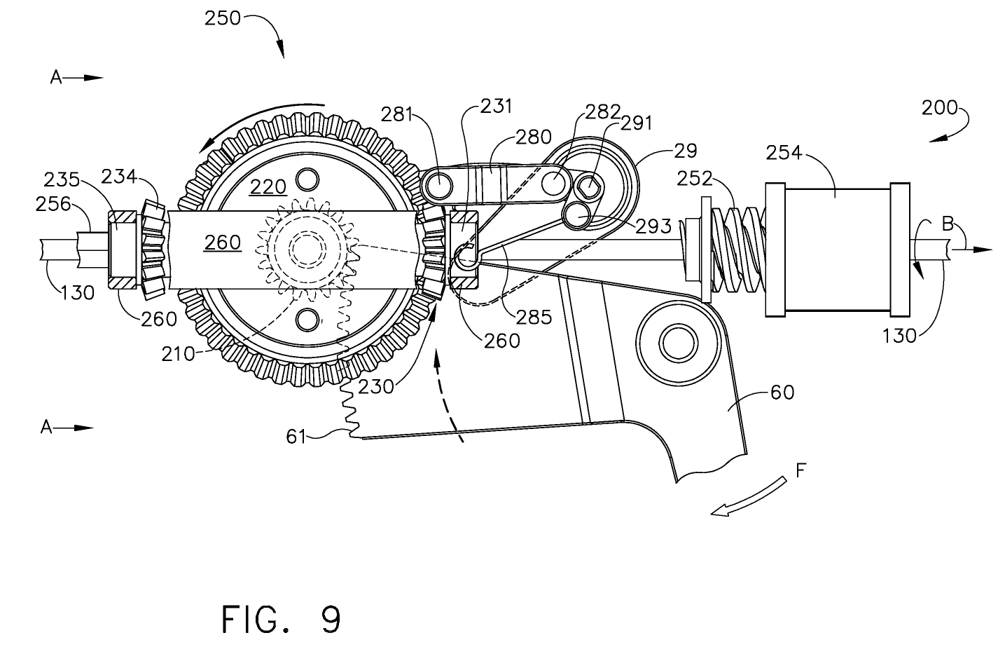

As shown in FIGS. 9-10, tissue cutting mechanism (200) of the present example is connected to trigger (28) by engagement of gear teeth (61) with a rotating spur gear (210). Actuation of trigger (28) engages teeth (61) with spur gear (210) to rotate gear (210) around a spur pin (212) that is operatively attached to left handle half (30). As shown in FIG. 4, movement of the trigger (28) from an un-actuated position to an actuated position (see directional arrow located between trigger (28) and pistol grip (27)) results in clockwise rotation of spur gear (210). As trigger (28) is released, the vacuum generated by vacuum pump (310) and/or the resilience of torsion spring (500) assists in returning trigger (28) to the un-actuated position, and spur gear (210) reverses directions and rotates counter-clockwise.

A one-way ratchet (218) is located between spur gear (210) and a large bevel gear (220). Spur gear (210) and bevel gear (220) are separate, and both rotate around spur pin (212). One-way ratchet (218) engages spur gear (210) with bevel gear (220) as trigger (28) is activated, and disengages spur gear (210) from bevel gear (220) when handle (28) is released. In operation, one-way ratchet (218) rotates bevel gear (220) clockwise as the operator pulls trigger (28) closed; and as the operator releases trigger (28), one way ratchet (218) disengages from the counterclockwise rotating spur gear (210), and bevel gear (220) becomes stationary. By way of example only, one-way ratchet (218) can be a simple dog clutch mechanism (not shown) with opposing sawtooth shaped teeth on each gear (210, 220) respectively, with the teeth intermeshing around spur pin (212) to drive in one rotational direction (around spur pin (212)) and to slip in the opposite direction. The teeth of such a dog clutch mechanism can be beveled on one side to spread gears (210, 220) apart to slip when rotated in the opposite rotational direction. A spring (not shown) can be placed around spur pin (212) (e.g., between left cover (30) and spur gear (210)) and used to normally bias spur gear (210) and bevel gear (220) together to drivingly engage the dog clutch mechanism. Other embodiments of a one-way ratchet (218) can include but are not limited to a ratchet and pawl, a sprag clutch, or a one way torsion spring encircling a pin to grip in one rotational direction and to release in the opposite rotational direction. Other suitable ratcheting mechanisms, clutching mechanisms, or other features or configurations, will be apparent to those of ordinary skill in the art in view of the teachings herein. Alternatively, spur gear (210) and bevel gear (220) may be unitary in some versions.

Referring to FIG. 4, hand actuation of trigger (28) rotates spur gear (210) clockwise, engages one way ratchet (218), and rotates bevel gear (220) clockwise. Rotary motion of bevel gear (220) is then transferred to cutter (130) by rotationally engaging a distal bevel gear (230) (FIG. 7) with large bevel gear (220). The rotational direction and translational direction of cutter (130) changes depending on whether proximal bevel gear (230) or distal bevel gear (234) is engaged with large bevel gear (220). A shift mechanism (250) is provided to change rotational direction and translational direction of cutter (130) in response to movement of directional reversal lever (29).

Referring to FIG. 7, trigger (28) is being actuated by the operator (not shown) and is moving toward pistol grip (27). Spur gear (210) is rotating (on far side of bevel gear (220)), and one-way ratchet (218) engages spur gear (210) with bevel gear (220) such that bevel gear (220) rotates counterclockwise (see arrow on gear (220) in FIG. 7) with spur gear (210). Directional reversal lever (29) is in the first position X (FIG. 1, 4, 7, 9), which brings distal bevel gear (230) of shift mechanism (250) into rotational and driving contact with bevel gear (220). Rotational movement of distal bevel gear (230) delivers rotational and translational movement to the cutter (130) in response to actuation of trigger (28) by the operator.

1. Exemplary Shift Mechanism

Shift mechanism (250) of the present example is best shown in FIGS. 7-10, and is operable to reverse the rotational and translational movement of cutter (130) in response to a positional change of reversal lever (29). As shown in FIG. 8, shift mechanism (250) includes cutter (130) and an externally threaded screw (252) that is fixedly attached thereto by over-molding, adhesives, or any other method of attachment. Externally threaded screw (252) is configured to threadably engage within an internally threaded stationary nut (254) of shift mechanism (250). Internally threaded stationary nut (254) is cylindrical in shape and is received in and constrained by left and right covers (30, 31). With internally threaded stationary nut (254) captured in left and right covers (30, 31), rotation of cutter (130) rotates externally threaded screw (252) within threaded stationary nut (254), and threaded engagement moves cutter (130) and externally threaded screw (252) longitudinally. Counter-clockwise rotation of hollow cutter (130) moves hollow cutter (130) proximally, away from piercing tip (102), to open tissue aperture (105); and clockwise rotation translates hollow cutter (130) distally, toward piercing tip (102), to close tissue aperture (105). All rotational directions of hollow cutter (130) in this example are taken from the view A-A

As shown in FIG. 8, shift mechanism (250) further comprises proximal bevel gear (234), distal bevel gear (230), and a cutter driver (256) fixedly attached to the hollow cutter (130) by a process such as overmolding or adhesion. Cutter driver (256) is spaced proximally from externally threaded screw (252) and further comprises a hexagonal drive portion (257) with a hexagonal cross section. Cutter driver (256) is configured to slidingly mount within a hex bore (232) at a center of distal gear (230) and a proximal hex bore (236) at a center of proximal gear (234). Hexagonal bores (232, 236) are configured to slide on and rotatably engage with hexagonal drive portion (257) of the cutter driver (256). Thus, cutter (130) rotates unitarily with bevel gears (230, 234) in this example, while being permitted to translate longitudinally relative to bevel gears (230, 234).

Distal gear (230) also comprises a distal bearing (231) configured to rotatably mount within a distal opening (258) from the inside of a shift fork (260); and proximal gear (234) has a proximal bearing (235) configured to rotatably mount (from the inside) within a proximal opening (259) within shift fork (260). Both gears (230, 234) are secured longitudinally inside of the “C” shape of shift fork (260) by a spacer (270) sized to fit between mounted gears (230, 234). Spacer (270) is shown as attached to shift fork (260) but can be separate piece that is placed over the cutting needle (30) between gears (230, 234) mounted in shift fork (260). Cutter (130) and cutter driver (256) is inserted through the proximal end of shift fork (260), through distal and proximal hex bores (232, 236) of gears (230, 234), and through the distal end of shift fork (260) to slidingly secure the assembly together within shift fork (260). Longitudinal movement of shift fork (260) (in either direction) moves the assembly of proximal and distal bevel gears (234, 230) and shift fork 260 together along hexagonal drive portion (257) of cutter driver (256).

Shift fork (260) is operably coupled to directional reversal lever (29) by a shift rod (280). A first pin (281) pivotally connects a proximal forked end of shift rod (280) to a tab (262) of shift fork (260); and a second pin (282) pivotally connects a distal end of shift rod (280) to a clevis (290) in a toggle rod (291). Toggle rod (291) attaches to directional reversal lever (29) and rotates in response to movement of directional reversal lever (29). Movement of directional reversal lever (29) rotates toggle rod (291), engages shift rod (280), and moves shift fork (260) longitudinally within handle halves (30, 31) to engage trigger (28) to cutter (130) through either proximal bevel gear (234) or distal bevel gear (230). An over-center leaf spring (285) is pivotally attached at one end (287) to a flange (292) of toggle rod (291) by pin (293). A second end (286) of over-center leaf spring (285) is pivotally attached to a pin (295) in right cover (31). Over-center leaf spring (285) biases (and holds) directional reversal lever (29) (and shift mechanism (250)) at one of either the X position or the Y position.

2. Exemplary Operation of the Shift Mechanism at Position X

The operation of shift mechanism (250) is best shown in FIGS. 9-10, wherein shift mechanism (250) and portions of tissue cutting mechanism (200) are shown with covers (30, 31) and trigger cover (68) removed. In FIG. 9, directional reversal lever (29) is in the first position X, and shift rod (280) has moved shift fork (260) proximally to bring distal bevel gear (230) into operative contact with bevel gear (220). In this view, a force F is being applied to inner member (60) of trigger (28) to rotatingly engage gear teeth (61) with spur gear (210). Ratchet (218) is rotating bevel gear (220) counter-clockwise (see arrow), and the toothed engagement of distal bevel gear (230) with bevel gear (220) is rotating cutter driver (256) and cutter (130) in a clockwise direction as viewed from A-A. Proximal bevel gear (234) and externally threaded screw (252) are also rotating clockwise (with proximal bevel gear (234) essentially “freewheeling”). The clockwise rotation of externally threaded screw (252) in internally threaded nut (254) is translating screw (252), hollow cutter (130), and cutter driver (256) distally to the right as indicated by the arrow B. Gears (234, 230) are rotating and hexagonal cutter driver (256) is rotating and sliding longitudinally therethrough while being driven by distal bevel gear (230). Over-center leaf spring (285) is over center and is holding shift mechanism (250) in restraint at the position shown.

3. Exemplary Operation of the Shift Mechanism at Position Y

In FIG. 10, directional reversal lever (29) has been moved to position Y and shift rod (280) has moved shift fork (260) distally to bring proximal bevel gear (234) into operative contact with large bevel gear (220). In this view, the force F is being applied to inner member (60) of trigger (28) to rotatingly engage gear teeth (61) with spur gear (210), and to rotate bevel gear (220) counter-clockwise (see arrow). The engagement of proximal bevel gear (234) with bevel gear (220) rotates proximal bevel gear (234), which rotates cutter driver (256) and cutter (130) in a counter-clockwise direction as viewed from A-A. Rotating cutter (130) also rotates externally threaded screw (252) and distal bevel gear (230) counter-clockwise (with distal bevel gear (230) essentially “freewheeling”). The counter-clockwise rotation of externally threaded screw (252) in internally threaded nut (254) is translating the hollow cutter (130) and externally threaded screw (252) proximally to the left, as indicated by the arrow B in FIG. 10. Once again, over-center leaf spring (285) is over center and is holding shift mechanism (250) in restraint at the position shown.

It should be understood that tissue cutting mechanism (200) and shift mechanism (250) may be varied in a number of ways. By way of example only, either or both mechanisms (200, 250) may include electromechanical components, including but not limited to motors or solenoids. Either or both mechanisms (200, 250) may also include various alternative mechanical components, features, or methods of operation. Other suitable features, components, configurations, and methods of operation for tissue cutting mechanism (200) and shift mechanism (250) will be apparent to those of ordinary skill in the art in view of the teachings herein.

### III. Exemplary Operation of the Biopsy Device

As noted above, biopsy device (25) of the present example is a manually actuated and manually powered device. Manual actuation of trigger (28) simultaneously powers tissue cutting mechanism (200) and vacuum generating mechanism (300) to collect and store tissue samples within tissue collection chamber (150). As described below, one or more actuations of trigger (28) may be required to sever, collect, and store the tissue samples within biopsy device (25).

In one example of operation, biopsy device (25) can be provided to the surgeon or operator with cutter (130) in a distalmost position (e.g., closing off tissue aperture (105)). This position can be easily verified by visually looking at tissue aperture (105). With cutter (130) in a distalmost position, directional reversal lever (29) is moved to position “Y” (see FIG. 1) to configure tissue cutting mechanism (200) to retract cutter (130) proximally and open tissue aperture (105) in response to actuations of trigger (28). If cutter (130) is retracted into the needle portion (100) and tissue aperture (105) is open, the operator moves directional reversal lever (29) to position “X” (see FIG. 1) so that cutter (130) will move distally in response to trigger (28) actuations and advance distally along tissue aperture (105) to cut tissue. For the following description, cutter (130) is in a distalmost position and directional reversal lever (29) is moved to position “Y”.

With cutter (130) in a distalmost position and directional reversal lever (29) at position “Y”, the surgeon or operator places piercing tip (102) against tissue. Using visualization such as unassisted visualization, x-rays, ultrasound, MRI and the like, the operator inserts needle portion (100) into tissue and positions needle portion (100) adjacent to a suspect lesion or tumor (e.g., within a patient's breast or elsewhere). If desired, needle portion (100) can be rotated with knob (90) to better position or orient tissue aperture (105) adjacent to the tissue lesion. Once tissue aperture (105) is in position, the operator begins manually actuating or pumping trigger (28) to power biopsy device (25) and to acquire the tissue sample.

Referring now to the elements shown in FIGS. 4-6, with directional reversal lever (29) at position “Y”, the first actuation of trigger (28) toward pistol grip (27) moves pump (310) of vacuum generating mechanism (300) from the position of FIG. 5 to that of FIG. 6. The movement of pump (310) pulls piston (318) down and cylinder portion (316) up to create or draw a vacuum. The vacuum created by the manual actuation of trigger (28) is communicated through flexible hose (340) to the collection base (153), and to tissue sample container (155) of tissue collection chamber (150). The vacuum is then communicated from tissue collection chamber (150) into cutter lumen (131) of cutter (130). Vacuum from lumen (131) is then communicated to hollow cutter passage (106) and lateral passage (107) within the needle portion (100). With cutter (130) at the distalmost position, auto pressure regulator (370) is biased to a distalmost position, and air passage (372), lateral passage (107), and hollow cutter passage (106) are open to atmospheric pressure air (FIG. 11).

The actuations of trigger (28) also power tissue cutting mechanism (200) at the same time the actuations power vacuum generating mechanism (300). As trigger (28) is depressed, the movement of trigger (28) moves gear teeth (61) in an arc to rotate spur gear (210) around pin (212). In FIG. 4, one-way ratchet (218) drives bevel gear (220) clockwise; and in FIG. 7, one-way ratchet (218) drives bevel gear (220) counter-clockwise. In FIG. 7, as bevel gear (220) rotates counter-clockwise, distal bevel gear (230) is engaged with bevel gear (220) and is rotated clockwise around the longitudinal axis extending within a center of cutter (130). As distal bevel gear (230) rotates, it rotates cutter driver (256), and hence cutter (130), to retract cutter (130) proximally to open tissue aperture (105). In particular, as cutter (130) rotates, externally threaded screw (252) moves cutter (130) and the auto pressure regulator (370) proximally. When auto pressure regulator (370) moves proximally to position air passage (372) proximal to seal (96), the venting or delivery of air at atmospheric pressure (through air passage (372)) is shut off to lateral passage (107) and hollow cutter passage (106). Vacuum is now delivered to lateral passage (107) to draw tissue into tissue aperture (105).

After about three repeated manual actuations of trigger (28), cutter (130) moves to the distalmost position of FIG. 12, and vacuum pump (310) draws tissue into tissue aperture (105). At this point, a visual indicator, an auditory sound, or a hard stop can be provided to indicate the distalmost position of cutter (130) to inform the operator that directional reversal lever (29) needs to be moved to position “X” (see FIG. 1). With directional reversal lever (29) at position “X”, the operator continues to pump or actuate and release trigger (28) to both draw vacuum to pull tissue into tissue aperture (105) and to advance and rotate cutter (130) to sever the tissue extending therein.

As cutter (130) approaches the distalmost position of FIG. 11, air passage (372) of auto pressure regulator (370) opens again to vent or deliver air at atmospheric pressure to lateral passage (107) and to hollow cutter passage (106). Cutter (130) may continue to rotate to at least some degree without advancing further when cutter (130) has reached the distalmost position. When cutter (130) has completely severed the tissue sample protruding into tissue aperture (105), the air at atmospheric pressure is conducted into air passage (372) along lateral passage (107), through vacuum passages (108), into cutter passage (106), and into lumen (131) of cutter (130) to create a pressure gradient to push the severed tissue sample therein proximally into tissue sample container (150). The tissue sample is deposited on the tissue collection grid (152), and any fluid communicated through lumen (131) passes through grid (152). It will be appreciated that several tissue samples may be obtained from a patient without having to withdraw needle portion (100) from the patient. In other words, biopsy device (25) may be used to obtain multiple tissue samples with just a single insertion of needle portion (100) into a patient.

It should be understood that there are a variety of other ways in which biopsy device (25) may be operated. Such alternative methods of use may be performed using biopsy device (25) of the present example or using variations of biopsy device (25) of the present example. Various alternative methods of use will be apparent to those of ordinary skill in the art in view of the teachings herein.

Embodiments of the present invention have application in conventional endoscopic and open surgical instrumentation as well as application in robotic-assisted surgery.

Embodiments of the devices disclosed herein can be designed to be disposed of after a single use, or they can be designed to be used multiple times. Embodiments may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, embodiments described herein may be processed before surgery. First, a new or used instrument may be obtained and if necessary cleaned. The instrument may then be sterilized. In one sterilization technique, the instrument is placed in a closed an sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A biopsy device for acquiring one or more tissue samples from a patient, the biopsy device comprising:
a hollow needle configured to penetrate tissue, wherein the hollow needle defines a lumen, the hollow needle having a tissue receiving port in communication with the lumen; and
a hand-held body, comprising:
a handle configured to be held in an operator's hand,
a vacuum pump in communication with the needle, wherein the vacuum pump is operable to draw tissue into the tissue receiving port,
a hollow tissue cutter movable within the lumen of the needle, wherein the tissue cutter is operable to sever tissue drawn into the tissue receiving port, wherein cutter defines lumen in communication with the vacuum pump, and
a hand actuated lever engaged with the handle for transferring motive power from the operator's hand to at least one of the vacuum pump or the cutter to acquire one or more severed tissue samples from the patient.

2. The biopsy device of claim 1, wherein the hand actuated lever is pivotally attached to the handle, wherein the hand actuated lever pivots from an open position to a closed position as the operator's hand closes, wherein the hand actuated lever pivots from the closed position to an open position as the operator's hand opens.

3. The biopsy device of claim 2, further comprising at least one gear tooth configured to transfer motive power from the hand actuated lever to at least one of the vacuum pump or the tissue cutter as the hand actuated lever pivots from an open position to a closed position.

4. The biopsy device of claim 2, further comprising at least one pivoting linkage configured to transfer motive power from the hand actuated lever to at least one of the vacuum pump or the tissue cutter as the hand actuated lever pivots from an open position to a closed position.

5. The biopsy device of claim 2, wherein the vacuum pump has an enclosed volume that expands to create a negative pressure to draw tissue into the tissue receiving port as the hand actuated lever pivots from an open position to a closed position.

6. The biopsy device of claim 5, wherein at least a portion of the vacuum pump moves linearly as the hand actuated lever pivots from the open position to the closed position to create the negative pressure within the vacuum pump.

7. The biopsy device of claim 6, wherein the vacuum pump comprises a hollow cylinder having an open end and a closed end and a movable piston within the cylinder, the piston being movable toward and away from the closed end and having a piston face facing the closed end, wherein an enclosed volume is defined within the cylinder between the piston face and the closed end.

8. The biopsy device of claim 5, wherein the vacuum pump generates a negative pressure between about 18 inches of mercury and about 20 inches of mercury.

9. The biopsy device of claim 2 further comprising a cutting mechanism configured to convert pivoting movement of the hand actuated lever into rotary movement of the tissue cutter as the hand actuated lever pivots from an open position to the closed position.

10. The biopsy device of claim 9 wherein the cutting mechanism further comprises at least one screw thread attached to the tissue cutter to convert the rotary movement of the tissue cutter into longitudinal translation of the tissue cutter.

11. The biopsy device of claim 10, wherein when the tissue cutter rotates in a first direction, the tissue cutter longitudinally translates proximally to open the tissue receiving port, and when the tissue cutter rotates in a second opposite direction, the tissue cutter longitudinally translates distally to close the tissue receiving port and to sever tissue drawn within.

12. The biopsy device of claim 11, wherein the cutting mechanism further comprises a shift mechanism that is movable between a first shift position and a second shift position, wherein the cutting mechanism is operable to rotate the tissue cutter in the first direction to open the tissue receiving port when the shift mechanism is in the first shift position, wherein the cutting mechanism is operable to rotate the tissue cutter in the second opposite direction to close the tissue receiving port and to sever tissue when the shift mechanism is in the second shift position.

13. The biopsy device of claim 12, wherein the shifting mechanism comprises a first gear and a second gear, wherein the shifting mechanism selectively shifts operative engagement of the hand actuated lever from one of the first gear or the second gear to change the direction of rotation of the tissue cutter.

14. The biopsy device of claim 1, wherein the biopsy device further comprises a tissue sample holder configured to receive severed tissue samples, wherein the tissue sample holder is in communication with the vacuum pump to draw severed tissue samples along the cutter lumen and into the tissue sample holder.

15. The biopsy device of claim 14, wherein the biopsy device further comprises a pressure regulator, wherein when vacuum is applied to a proximal end of the cutter lumen and the tissue receiving port is closed, the pressure regulator is configured to vent a distal portion of the hollow of the needle to push the severed tissue sample along the cutter lumen and into the tissue sample holder.

16. A biopsy device for acquiring one or more tissue samples from a patient, the biopsy device comprising:
a hollow needle configured to penetrate tissue, wherein the hollow needle defines a needle lumen, the hollow needle having a tissue receiving port in communication with the needle lumen, the tissue receiving port being transverse to the needle lumen; and
a hand-held body, comprising:
a pistol grip configured to be held in an operator's hand,
a hollow tissue cutter movable within the needle lumen, wherein the tissue cutter is operable to sever tissue drawn into the tissue receiving port, wherein cutter defines cutter lumen,
a vacuum pump in communication with the cutter lumen, wherein the vacuum pump is operable to draw tissue into the tissue receiving port via the cutter lumen,
a cutter actuation mechanism, wherein the cutter actuation mechanism is manually operable perform one or both of translating the cutter or rotating the cutter, and
a hand actuated lever engaged with the pistol grip, wherein the hand actuated lever is in communication with the vacuum pump and the cutter actuation mechanism, wherein the hand actuated lever is manually operable to simultaneously actuate the vacuum pump and the cutter actuation mechanism.

17. The biopsy device of claim 16, further comprising a tissue collection chamber removably coupled with the hand-held body, wherein the vacuum pump is in communication with the cutter lumen via the tissue collection chamber.

18. A method of acquiring one or more tissue samples from a patient comprising:
providing a biopsy device, wherein the biopsy device comprises:
a tissue penetrating needle having a tissue receiving port,
a hand-held body, wherein the needle extends distally from the body, wherein the body comprises:
a vacuum pump in communication with the needle, wherein the vacuum pump is operable to draw tissue into the tissue receiving port,
a hollow tissue cutter having a cutting edge, wherein the tissue cutter is disposed within the needle, the tissue cutter being movable within the needle for cutting tissue drawn into the tissue receiving port, and
a hand lever movable from an open position to a closed position;
inserting the needle into a patient's breast;
manually moving the hand lever from the open position to the closed position to apply motive power to the vacuum pump, wherein the motive power delivered to the vacuum pump creates a negative pressure in the vacuum pump to draw tissue into the tissue receiving port; and
removing the needle from the patient's breast.

19. The method of claim 18, wherein the act of manually moving the hand lever from the open position to the closed position actuates the tissue cutter by moving the tissue cutter longitudinally to sever tissue drawn into the tissue receiving port.

20. The method of claim 19, further comprising manually moving the hand lever from the open position to the closed position a plurality of times to obtain a plurality of tissue samples, wherein the acts of manually moving the hand lever from the open position to the closed position a plurality of times is performed before the act of removing the needle from the patient's breast.
